(19) United States
(12) Patent Application Publication
(10) Pub. No.: US 2023/0414865 A1
(43) Pub. Date: Dec. 28, 2023

(54) WEARABLE DRUG DELIVERY DEVICE WITH INCORPORATED FILTRATION

(71) Applicant: INSULET CORPORATION, Acton, MA (US)

(72) Inventors: Jianhong ZHAO, Carlisle, MA (US); Soroush KAMRAVA, Everett, MA (US); James CAUSEY, Simi Valley, CA (US); Daniel BARRETT, Somerville, MA (US); Craig BRODEUR, Marlborough, MA (US); Eric DUHAMEL, Boxborough, MA (US); Ann LIU, Lexington, MA (US); Ian MCLAUGHLIN, Groton, MA (US)

(21) Appl. No.: 18/340,339

(22) Filed: Jun. 23, 2023

Related U.S. Application Data
(60) Provisional application No. 63/354,813, filed on Jun. 23, 2022.

(51) Int. Cl. A61M 5/142; A61M 5/158; A61M 5/148

(52) U.S. Cl. CPC A61M 5/14248; A61M 5/158; A61M 5/148; A61M 2005/14252; A61M 2005/1585

(57) ABSTRACT

A drug delivery device including a reservoir operable to contain a liquid drug, a fluid outlet including an outlet reservoir opening and an outlet fluid channel, wherein the outlet reservoir opening has a volume and is fluidly coupled to the outlet fluid channel, and an outlet filter plug operable to fill the volume of the outlet reservoir opening and collect particulate material as the liquid drug is expelled from the reservoir.

### RELATED APPLICATIONS

This application claims the benefit of U.S. Provisional Patent Application No. 63/354,813, filed Jun. 23, 2022, the entire contents of which are incorporated herein by reference in their entirety.

### TECHNICAL FIELD

The disclosed embodiments generally relate to medication delivery. More particularly, the disclosed embodiments relate to techniques, processes, systems, and devices for filtering and delivering a medicament to a user.

### BACKGROUND

Many wearable drug delivery devices include a reservoir for storing a liquid drug. A drive mechanism is operated to expel the stored liquid drug from the reservoir for delivery to a user. However, the liquid drug may aggregate or precipitate over time in the reservoir. The aggregation or precipitation of the liquid drug can generate submicron particulates that may cause irritation and inflammation when delivered to the user, or may occlude the needle or cannula through which the liquid drug is delivered to the user.

Accordingly, there is a need for an improved system for storing and expelling a liquid drug from a reservoir, which also filters undesired particulates from the liquid drug.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

At least one aspect of the present disclosure is directed to a drug delivery device. The drug delivery device includes a reservoir operable to contain a liquid drug, a fluid outlet including an outlet reservoir opening and an outlet fluid channel, wherein the outlet reservoir opening has a volume and is fluidly coupled to the outlet fluid channel, and an outlet filter plug operable to fill the volume of the outlet reservoir opening and collect particulate material as the liquid drug is expelled from the reservoir.

In one or more embodiments, the outlet filter plug includes a porous material having a pore size of about 1 micrometer to about 10 micrometers. In some embodiments, the outlet filter plug includes a porous material having a pore size of about 1 micrometer to about 10 micrometers. In various embodiments, the outlet filter plug may be operable to absorb phenolic preservatives in the liquid drug. In certain embodiments, the outlet filter plug is operable to collect precipitant in the liquid drug.

In some embodiments, the outlet filter plug includes a porous silica material, a porous alumina material, a porous polymeric material, or a combination thereof. In an embodiment, the drug delivery device includes a hard cannula or needle coupled to the outlet fluid channel after the outlet filter plug. The hard cannula or needle may be a fluid pathway for the liquid drug after the liquid drug has passed through the outlet filter plug. In certain embodiments, the drug delivery device includes a duckbill valve disposed within the hard cannula or needle. The duckbill valve may have a cracking pressure operable to allow a hexamer form of the liquid drug to pass to the distal tip of the hard cannula or needle. In various embodiments, the duckbill valve is positioned proximate a distal end of the hard cannula.

In an embodiment, the drug delivery device includes a fluid inlet including an inlet septum, an inlet fluid channel, and an inlet reservoir opening, wherein the inlet reservoir opening has an inlet volume and is fluidly coupled to the reservoir, and an inlet filter plug operable to fill the volume of the inlet reservoir opening and collect particulate material in the liquid drug entering via the inlet fluid channel prior to entering the reservoir. In some embodiments, the fluid inlet includes an inlet septum operable to seal the inlet fluid channel from an exterior of the drug delivery device. The inlet filter plug may be operable to absorb phenolic preservatives in the liquid drug. The inlet filter plug may be operable to collect precipitant in the liquid drug.

Another aspect of the present disclosure is directed to a drug delivery device that includes a reservoir operable to contain a liquid drug, a fluid outlet including an outlet reservoir opening and an outlet fluid channel, a filtration module having an inlet, a filter and an outlet, and a needle coupled to the outlet fluid channel and operable to receive the liquid drug when the liquid drug is expelled from the reservoir. The needle may have a first segment operable to fluidly couple to the inlet and a second segment that may be operable to couple the outlet of the filtration module, and the filter is comprised of a porous material. The porous material may be a porous silica material, a porous alumina material, a porous polymeric material, or a combination thereof.

In some embodiments, the porous material may have a pore size of about 1 micrometer to about 10 micrometers. The inlet of the filtration module may include a pierceable inlet septum. The outlet of the filtration module may include a pierceable outlet septum. The needle may have a first segment operable to pierce the inlet septum and a second segment operable to pierce the outlet septum.

### BRIEF DESCRIPTION OF THE DRAWINGS

In the drawings, like reference characters generally refer to the same parts throughout the different views. In the following description, various embodiments of the present disclosure are described with reference to the following drawings, in which:

FIG. 1 illustrates a schematic diagram of a drug delivery system according to embodiments of the present disclosure;

FIG. 2A illustrates a schematic diagram of a drug delivery system according to embodiments of the present disclosure;

FIG. 2B illustrates a perspective view of an example of a drug delivery system of FIG. 2A according to embodiments of the present disclosure;

FIG. 3 illustrates a reservoir operable for use in a drug delivery system according to embodiments of the present disclosure;

FIG. 4 illustrates a filter arrangement operable for use in a drug delivery system according to embodiments of the present disclosure;

FIG. 5A illustrates a filter arrangement operable for use in a drug delivery system according to embodiments of the present disclosure;

FIG. 5B illustrates a filter arrangement operable for use in a drug delivery system according to embodiments of the present disclosure;

FIG. 5C illustrates an overhead view of the filter arrangement of FIG. 5A according to embodiments of the present disclosure;

FIG. 6 illustrates a filter arrangement operable for use in a drug delivery system according to embodiments of the present disclosure;

FIG. 7A illustrates a flow director arrangement operable for use in a drug delivery system according to embodiments of the present disclosure; and

FIG. 7B illustrates operation of a duckbill valve included in the flow director arrangement of FIG. 7A.

FIG. 8A illustrates an in-line radial flow cartridge filter according to embodiments of the present disclosure.

FIG. 8B illustrates an in-line axial flow cartridge filter according to embodiments of the present disclosure.

FIG. 8C illustrates in-line radial and axial flow cartridge filters according to embodiments of the present disclosure prior to addition of adsorption media, wherein each 1 is independently a support structure, 2 is a pre-filter or post/particulate filter, and 7 is empty fluid cavity, or open cell PVA foam.

FIG. 8D illustrates in-line radial flow cartridge filter according to embodiments of the present disclosure, wherein each 1 is independently a support structure, 2 is a pre-filter or post/particulate filter, 3 is an adsorption media, and 4 is a bubble vent.

FIG. 8E illustrates in-line radial and axial flow cartridge filters according to embodiments of the present disclosure prior to addition of adsorption media, wherein each 1 is independently a support structure, 2 is a pre-filter or post/particulate filter, 4 is a bubble vent, and 7 is empty fluid cavity, or open cell PVA foam.

FIG. 9A illustrates reservoir integration filter according to embodiments of the present disclosure, wherein each 1 is independently a support structure, 2 is a pre-filter or post/particulate filter, and 3 is an adsorption media.

FIG. 9B illustrates reservoir integration filter according to embodiments of the present disclosure prior to addition of adsorption media, wherein each 1 is independently a support structure and 2 is a pre-filter or post/particulate filter.

FIG. 9C illustrates reservoir integration filter according to embodiments of the present disclosure, wherein each 1 is independently a support structure and 2 is a pre-filter or post/particulate filter.

FIG. 9D illustrates reservoir integration filter according to embodiments of the present disclosure, wherein each 1 is independently a support structure, 2 is a pre-filter or post/particulate filter, 3 is an adsorption media, and 4 is a bubble vent.

FIG. 9E illustrates reservoir integration filter according to embodiments of the present disclosure prior to addition of adsorption media, wherein each 1 is independently a support structure, 2 is a pre-filter or post/particulate filter, and 4 is a bubble vent.

FIG. 9F illustrates reservoir integration filter according to embodiments of the present disclosure, wherein each 1 is independently a support structure, 2 is a pre-filter or post/particulate filter, 3 is reservoir additives and 4 is a bubble vent.

FIG. 10 illustrates porous sintered polymer or mixed matrix membrane filter according to embodiments of the present disclosure, wherein each 1 is independently a support structure and 2 is a porous sintered polymer or mixed matrix membrane.

FIG. 11 illustrates reservoir interaction filter according to embodiments of the present disclosure, wherein each 1 is independently a support structure, 2 is a pre-filter or post/particulate filter, and 3 is an adsorption media.

FIG. 12 illustrates reservoir interaction filter according to embodiments of the present disclosure, wherein each 1 is independently a support structure, 2 is a pre-filter or post/particulate filter, and 3 is an adsorption media.

The drawings are not necessarily to scale. The drawings are merely representations, not intended to portray specific parameters of the disclosure. The drawings are intended to depict exemplary embodiments of the disclosure, and therefore are not to be considered as limiting in scope. Furthermore, certain elements in some of the figures may be omitted, or illustrated not-to-scale, for illustrative clarity. Still furthermore, for clarity, some reference numbers may be omitted in certain drawings.

### DETAILED DESCRIPTION

Systems, devices, and methods in accordance with the present disclosure will now be described more fully hereinafter with reference to the accompanying drawings, where one or more embodiments are shown. The systems, devices, and methods may be embodied in many different forms and are not to be construed as being limited to the embodiments set forth herein. Instead, these embodiments are provided so the disclosure will be thorough and complete, and will fully convey the scope of methods and devices to those skilled in the art. Each of the systems, devices, and methods disclosed herein provides one or more advantages over conventional systems, components, and methods.

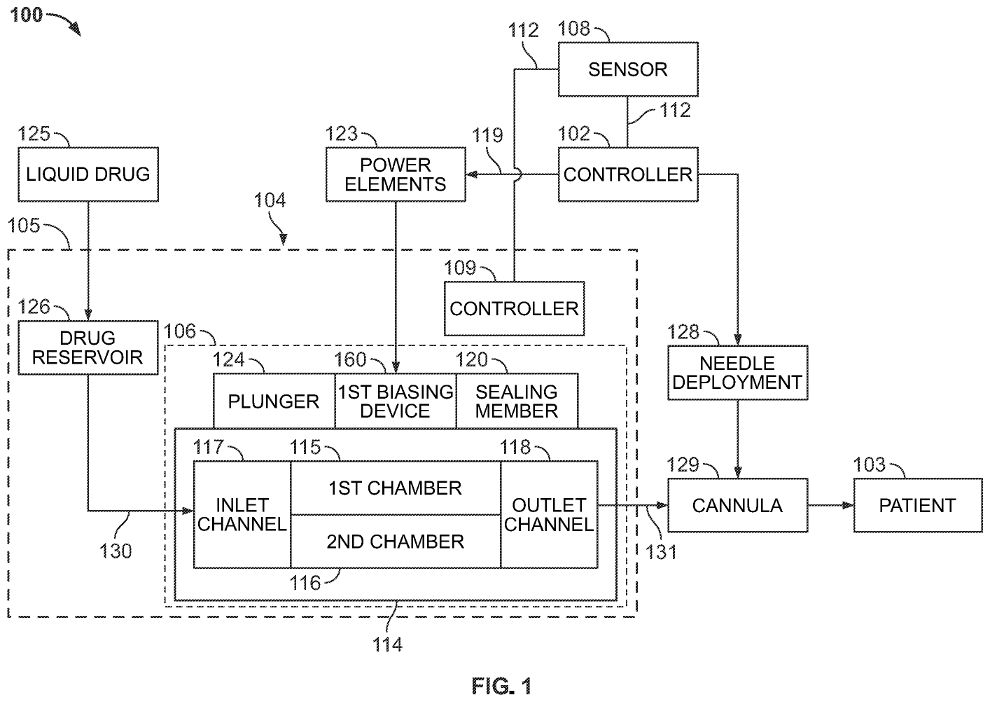

FIG. 1 illustrates a simplified block diagram of an example system 100. The system 100 may be a wearable or on-body drug delivery device and/or an analyte sensor attached to the skin of a patient 103. The system 100 may include a controller 102, a pump mechanism 104 (hereinafter “pump 104”), and a sensor 108. The sensor 108 may be one or more of a glucose or other analyte monitor such as, for example, a continuous glucose monitor, a ketone sensor, a heart rate monitor, a blood oxygen sensor element, or the like, and may be incorporated into the wearable device. The sensor(s) 108 may, for example, be operable to measure blood glucose (BG) values of a user to generate a measured BG level signal 112. The controller 102, the pump 104, and the sensor(s) 108 may be communicatively coupled to one another via a wired or wireless communication path. For example, each of the controller 102, the pump 104 and the sensor(s) 108 may be equipped with a wireless radio frequency transceiver operable to communicate via one or more communication protocols, such as Bluetooth®, or the like. As will be described in greater detail herein, the system 100 may also include a pump 104 which includes a drive mechanism 106 having at least one housing 114 defining a pump chamber 115, a channel chamber 116, an inlet channel 117, and an outlet channel 118. The drive mechanism 106 may further include a resilient sealing member 120 enclosing the pump chamber 115, and a first biasing device 160 (e.g., shape memory alloy (SMA) wire or the like) operable to bias or move a plunger 124 relative to the pump chamber 115, as will be described in greater detail herein. In an example, the drive mechanism 106, the controller 109 and the drug reservoir 126 may be incorporated in a housing 105, which may be one or more parts that may be a combination of reusable and disposable parts. The housing 105 may be formed from one or more materials, such as a plastic, a metal or the like. The system 100 may include additional components not shown or described for the sake of brevity.

The controller 102 may receive a desired BG level signal, which may be a first signal, indicating a desired BG level or range for the patient 103. The desired BG level or range may be stored in memory of a controller 109 on pump 104, received from a user interface of the controller 102, or another device, or by an algorithm within controller 109 (or controller 102). The sensor(s) 108 may be coupled to the patient 103 and operable to measure an approximate value of a BG level of the user. In response to the measured BG level or value, the sensor(s) 108 may generate a signal indicating the measured BG value. As shown in the example, the controller 102 may also receive from the sensor(s) 108 via a communication path, the measured BG level signal 112, which may be a second signal.

Based on the desired BG level and the measured BG level signal 112, the controller 102 or controller 109 may generate one or more control signals for directing operation of the pump 104. For example, one control signal 119 from the controller 102 or controller 109 may cause the pump 104 to turn on, or activate one or more power elements 123 operably connected with the pump 104. In the case where the first biasing device 160 is an SMA wire, activation of the SMA wire by the power element 123 may cause the SMA wire to change shape and/or length, which in turn may cause movement of the plunger 124 and the resilient sealing member 120. The specified amount of a liquid drug 125 (e.g., insulin, GLP-1, pramlintide, or a co-formulation of insulin, GLP-1 or pramlintide; a chemotherapy drug; a blood thinner; a pain medication; an arthritis drug; or the like) may be drawn from the reservoir 126 into the pump chamber 115, through the inlet channel 117, in response to a change in pressure due to the change in configuration of the resilient sealing member 120 and the plunger 124. In some examples, the specified amount of the liquid drug 125 to be delivered may be determined based on a difference between the desired BG level and the actual BG level signal 112. For example, specified amount of the liquid drug 125 may be determined as an appropriate amount of insulin to drive the measured BG level of the user toward the desired BG level. Based on operation of the pump 104, as determined by the control signal 119, the patient 103 may receive the liquid drug from a reservoir 126. The system 100 may operate as a closed-loop system, an open-loop system, or as a hybrid system. In an exemplary closed-loop system, the controller 109 may direct operation of the pump 104 without input from the user or controller 102, and may receive BG level signal 112 from the sensor(s) 108. The sensor(s) 108 may be housed within the pump 104 or may be housed in a separate device and communicate wirelessly directly with the pump 104 (e.g., with controller 109) or with an external controller 102.

As further shown, the system 100 may include a needle deployment component 128 in communication with the controller 102 or the controller 109. Though shown separately, needle deployment component 128 may be integrated within pump 104. The needle deployment component 128 may include a needle and/or cannula 129 deployable into the patient 103 and may have one or more lumens and one or more holes at a distal end thereof. The cannula 129 may form a portion of a fluid path coupling the patient 103 to the reservoir 126. More specifically, the inlet channel 117 may be coupled to the reservoir 126 by a first fluid path component 130. The first fluid path component 130 may be of any size and shape and may be made from any material. The first fluid path component 130 enables fluid, such as the liquid drug 125 in the reservoir 126, to be transferred to the drive mechanism 106.

As further shown, the outlet channel 118 may be coupled to the cannula 129 by a second fluid path component 131. The second fluid path component 131 may be of any size and shape and may be made from any material. The second fluid path component 131 may be connected to the cannula 129 to allow fluid expelled from the pump 104 to be provided to the patient 103. The first and second fluid path components 130 and 131 may be rigid, flexible, or a combination thereof.

The controller 102/109 may be implemented in hardware, software, or any combination thereof. The controller 102/109 may, for example, be a processor, a logic circuit or a microcontroller coupled to a memory. The controller 102/109 may maintain a date and time as well as provide other functions (e.g., calculations or the like) performed by processors. The controller 102/109 may be operable to execute an artificial pancreas (AP) algorithm stored in memory (not shown in this example) that enables the controller 102/109 to direct operation of the pump 104. For example, the controller 102/109 may be operable to receive an input from the sensor(s) 108, wherein the input comprises analyte level data, such as blood glucose data or levels over time. Based on the analyte level data, the controller 102/109 may modify the behavior of the pump 104 and resulting amount of the liquid drug 125 to be delivered to the patient 103.

The power elements 123 may be a battery, a supercapacitor, a piezoelectric device, or the like, for supplying electrical power to the pump 104. In other embodiments, the power element 123, or an additional power source (not shown), may also supply power to other components of the pump 104, such as the controller 102, memory, the sensor(s) 108, and/or the needle deployment component 128.

In an example, the sensor(s) 108 may be a device communicatively coupled to the controller 102 and may be operable to measure a blood glucose value at a predetermined time interval, such as approximately every 5 minutes, 1 minute, or the like. The sensor(s) 108 may provide a number of blood glucose measurement values to the AP application.

In some embodiments, the pump 104, when operating in a normal mode of operation, provides insulin stored in the reservoir 126 to the patient 103 based on information (e.g., blood glucose measurement values, target blood glucose values, insulin on board, prior insulin deliveries, time of day, day of the week, inputs from an inertial measurement unit, global positioning system-enabled devices, Wi-Fi-enabled devices, or the like) provided by the sensor(s) 108 or other functional elements of the system 100 or pump 104. For example, the pump 104 may contain analog and/or digital circuitry that may be implemented at the controller 102/109 for controlling the delivery of the drug or therapeutic agent. The circuitry used to implement the controller 102/109 may include discrete, specialized logic and/or components, an application-specific integrated circuit, a microcontroller or processor that executes software instructions, firmware, programming instructions or programming code enabling, for example, an AP application stored in memory, or any combination thereof. For example, the controller 102/109 may execute a control algorithm and other programming code that may make the controller 102/109 operable to cause the pump to deliver doses of the drug or therapeutic agent to a user at predetermined intervals or as needed to bring blood glucose measurement values to a target blood glucose value. The size and/or timing of some of the doses may be pre-programmed, for example, into the AP application by the patient 103 or by a third party (such as a health care provider, a parent or guardian, a manufacturer of the wearable drug delivery device, or the like) using a wired or wireless link, or may be calculated iteratively by the controller 102 or controller 109, such as every 5 minutes.

Although not shown, in some embodiments, the sensor(s) 108 may include a processor, memory, a sensing or measuring device, and a communication device. The memory of the sensor(s) 108 may store an instance of an AP application as well as other programming code and be operable to store data related to the AP application.

In various embodiments, the processor of the sensor(s) 108 may include discrete, specialized logic and/or components, an application-specific integrated circuit, a microcontroller or processor that executes software instructions, firmware, programming instructions stored in memory, or any combination thereof.

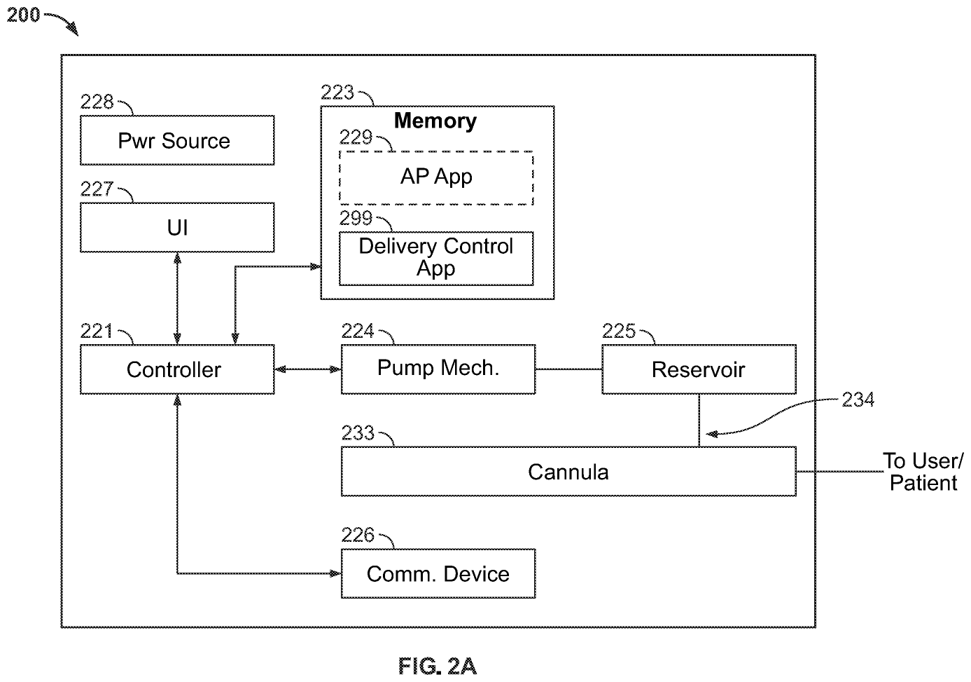

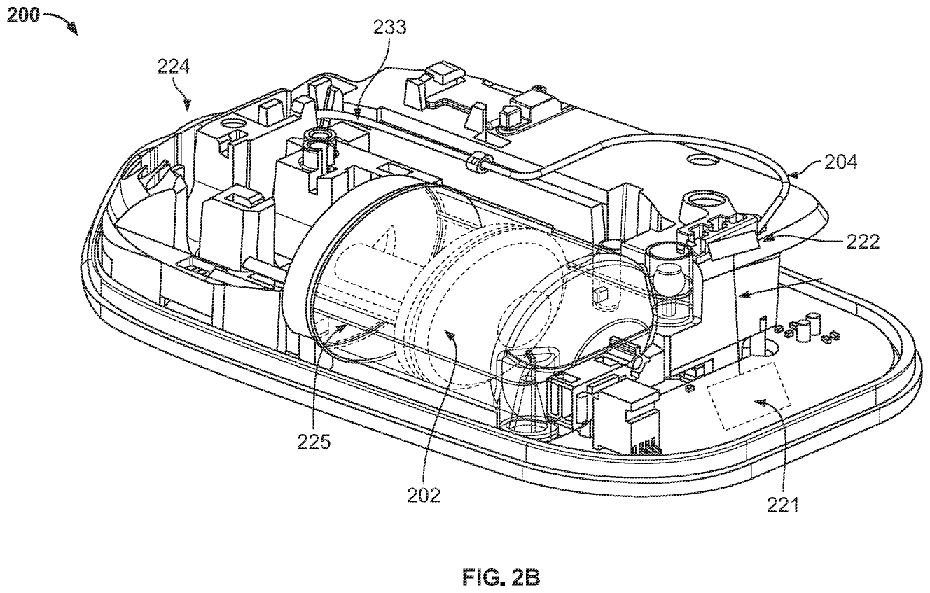

FIG. 2A illustrates a simplified block diagram of another example system 200. The system 200 may include a controller 221, a memory 223, an AP application 229 and delivery control application 299 stored in the memory 223, a pump mechanism 224, a communication device 226, user interface 227, and a power source 228. The memory 223 may be operable to store programming code and applications including a delivery control application 299, the AP application 229 and data. The delivery control application 299 and the AP application 229 may optionally be stored on other devices.

The controller 221 may be coupled to the pump mechanism 224 and the memory 223. The controller 221 may include logic circuits, a clock, a counter or timer as well as other processing circuitry, and be operable to execute programming code and the applications stored in the memory 223 including the delivery control application 299. A communication device 226 may be communicatively coupled to the controller 221 and may be operable to wirelessly communicate with an external device, such as a personal diabetes management device, a smart device such as a smartphone and/or a smartwatch, or the like.

The pump mechanism 224 may be operable to deliver a drug, like insulin, at a fixed or variable rate. For example, an AP application or AID algorithm executing on a personal diabetes management device or a smart phone may determine or be informed that a user's total daily insulin (e.g., bolus and/or basal deliveries) is 48 units per 24 hours, which may translate to an exemplary physiological dosage rate of 1 unit per hour that may be determined according to a diabetes treatment plan. However, the pump mechanism 224 may be operable to deliver insulin at rates different from the example physiological dosage rate of 1 unit per hour. In an example, the system 200 may be attached to the body of a user, such as a patient or diabetic via, for example, an adhesive, (e.g., directly attached to the skin of the user) and may deliver any therapeutic agent, including any drug or medicine, such as insulin, morphine, or the like, to the user. In an example, a surface of the system 200 may include an adhesive (not shown) to facilitate attachment to a user. The system 200 may, for example, be worn on a belt or in a pocket of the user and the liquid drug may be delivered to the user via tubing to an infusion site on the user.

In various examples, the system 200 may be an automatic, wearable drug delivery device. For example, the system 200 may include a reservoir 225 configured to hold a liquid drug (such as insulin), a needle and/or cannula 233 for delivering the drug into the body of the user (which may be done subcutaneously, intraperitoneally, or intravenously), and a pump mechanism 224, or other drive mechanism, for transferring the drug from the reservoir 225, through a needle or cannula 233, and into the user.

The pump mechanism 224 may be fluidly coupled to reservoir 225, and communicatively coupled to the medical device controller 221. The pump mechanism 224 may be coupled to the reservoir 225 and operable to output the liquid drug from the reservoir 225 via a fluid delivery path and out of the cannula 233. The pump mechanism 224 may have mechanical parameters and specifications, such as a pump resolution, that indicate mechanical capabilities of the pump mechanism. The pump resolution is a fixed amount of insulin the pump mechanism 224 delivers in a pump mechanism pulse, which is an actuation of the pump mechanism for a preset time period. Actuation may be when power from the power source 228 is applied to the pump mechanism 224 and the pump mechanism 224 operates to pump a fixed amount of insulin in a preset amount of time from the reservoir 225.

The cannula 233 of FIG. 2A may be coupled to the reservoir 225 via a fluid delivery path 234. The cannula 233 may be operable to output the liquid drug to a user when the cannula 233 is inserted in the user.

The system 200 may also include a power source 228, such as a battery, a supercapacitor, a piezoelectric device, or the like, that is operable to supply electrical power to the pump mechanism 224 and/or other components (such as the controller 221, memory 223, and the communication device 226) of the system 200.

As shown in FIG. 2B, the system 200 may include a plunger 202 positioned within the reservoir 225. An end portion or stem of the plunger 202 can extend outside of the reservoir 225. The pump mechanism 224 may, under control of the controller 221, be operable to cause the plunger 202 to expel the fluid, such as a liquid drug (not shown) from the reservoir 225 and into a fluid component 204 and cannula 233 by advancing into the reservoir 225. In various examples, a pressure sensor, such as that shown at 222, may be integrated anywhere along the overall fluid delivery path of the system 200, which includes the reservoir 225, the fluid delivery path component 204, and the cannula 233.

The controller 221 may be implemented in hardware, software, or any combination thereof. In various examples, the controller 221 can be implemented as dedicated hardware (e.g., as an application specific integrated circuit (ASIC)). The controller 221 may be a constituent part of the system 200, can be implemented in software as a computational model, or can be implemented external to the system 200 (e.g., remotely). The controller 221 may be configured to communicate with one or more sensors (e.g., sensor(s) 108 of FIG. 1).

As described above, a reservoir, such as 225, may be included in a drug delivery device to store a liquid drug (e.g., insulin). For example, the reservoir 225 may be filled, or partially filled, with a liquid drug or a liquid drug solution. In one example, a liquid drug solution is a mixture of the liquid drug and added preservatives. The reservoir may store the liquid drug until all of the liquid drug has been dispensed (e.g., into a patient via a cannula). As such, the liquid drug (or solution) may remain in the reservoir for a period of time (e.g., 1 day, 3 days, 1 week, 2 weeks, etc.). In some cases, the liquid drug (or solution) may aggregate or precipitate over time in the reservoir. The aggregation or precipitation of the liquid drug can generate submicron particulates that may cause irritation and inflammation when delivered to the patient or may cause an occlusion somewhere along the fluid path.

Accordingly, improved drug delivery devices with incorporated filtration are provided herein. In at least one embodiment, the drug delivery device includes at least one filter configured to collect particulate material as the liquid drug (or solution) is expelled from the reservoir. In one example, the at least one filter is a filter plug operable to fill the volume of an outlet opening and/or an inlet opening of the reservoir. In some examples, the at least one filter is included in a filtration module that is fluidly coupled to the reservoir 225.

In some embodiments, a filter of a drug delivery device comprises an absorption media, and optionally a pre-filter and a post filter. In some embodiments, a filter of a drug delivery device may further comprise a support structure. In some embodiments, a filter of a drug delivery device comprises an adsorption media positioned between a pre-filter and a post-filter. In some embodiments, a filter of a drug delivery device comprises a support structure, an adsorption media, a pre-filter, and a post-filter, wherein the adsorption media is positioned between the pre-filter and the post-filter. In some embodiments, a pre-filter or a post filter of a filter of a drug delivery device may have a maximum pore size that is smaller than the minimum particle size of an adsorption media. In some embodiments, a pre-filter or a post filter of a filter of a drug delivery device independently comprises cellulose acetate, PVDF, or PES.

In some embodiments, a filter of a drug delivery device comprises a feature that prevents air getting trapped between membranes. In some embodiments, a filter of a drug delivery device comprises a bubble vent. In some embodiments, a bubble vent is a hydrophobic vent. In some embodiments, a bubble vent is a ePTFE membrane.

In one example, a filter of a drug delivery device is made from one or more porous filtration materials. In some examples, the porous filtration material may be a silica material, an alumina material, a polymeric material, or any combination thereof. For example, the porous filtration material of a filter of a drug delivery device may be a modified silica/alumina material. In some embodiments, an adsorption media comprises zeolite. In some embodiments, zeolite may have a controlled minimum particle size to prevent migration. In some embodiments, zeolite may be a zeolite membrane, a zeolite pellet, or a porous structure containing zeolite. In some embodiments, zeolite is ultra-stable zeolite Y. In some embodiments, an adsorption media comprises one or more chelators. In some embodiments, an adsorption media comprises EDTA, EGTA, HEDTA, NTA or combination thereof. In some embodiments, an adsorption media comprises EDTA.

In some embodiments, a filter of a drug delivery comprises one or more porous sintered polymers or a mixed media membranes. In some embodiments, a filter of a drug delivery comprises a one or more porous sintered polymers or a mixed media membranes and further comprises at least one support structure. In some embodiments, a filter of a drug delivery comprises a one or more porous sintered polymers or a mixed media membranes and further comprises at least two support structures. In some embodiments, zeolite may be integrated into one or more porous sintered polymers or mixed matrix membranes. In some embodiments, a porous sintered polymers comprises sintered polymer resin. In some embodiments, a sintered polymer resin is sintered polyvinylidene difluoride (PVDF), sintered polyether sulfone (PES), or sintered polyether ether ketone (PEEK). In some embodiments, sintered polymer resin is sintered polyvinylidene difluoride (PVDF).

In some examples, the porous filtration material of a filter of a drug delivery device may be rigid bare inorganic material In some examples, the porous filtration material of a filter of a drug delivery device may be a sponge-like polymeric material with a pore size of about 1 to 10 μm. In some embodiments, a filtration material of a filter of a drug delivery device may have a pore size of about 0.2 μm to 10.0 μm. In some embodiments, a filtration material of a filter of a drug delivery device may have a pore size of about 0.2 μm to 5.0 μm. In some embodiments, a filtration material of a filter of a drug delivery device may have a pore size of about 5.0 μm to 10.0 μm. In some embodiments, a filtration material of a filter of a drug delivery device may have a pore size of about 2.5 μm to 7.5 μm. In some embodiments, a filtration material of a filter of a drug delivery device may have a pore size of about 0.2 μm. In some embodiments, a filtration material of a filter of a drug delivery device may have a pore size of about 0.5 μm. In some embodiments, a filtration material of a filter of a drug delivery device may have a pore size of about 1.0 μm. In some embodiments, a filtration material of a filter of a drug delivery device may have a pore size of about 2.5 μm. In some embodiments, a filtration material of a filter of a drug delivery device may have a pore size of about 5.0 μm. In some embodiments, a filtration material of a filter of a drug delivery device may have a pore size of about 7.5 μm. In some embodiments, a filtration material of a filter of a drug delivery device may have a pore size of about 10.0 μm.

In one example, the porous filtration material may collect liquid drug aggregates or precipitants (e.g., insulin fibrils) that form over time in the liquid drug solution. In some examples, the porous filtration material may act to absorb preservatives in the liquid drug (or solution). A filter of a drug delivery device may prevent undesired particulates and/or preservatives from being delivered to the patient. In some embodiments, undesired particulates and/or preservatives may comprise zinc, m-cresol, phenol, or methyl-hydroxy benzoate. In addition, by filtering out large particulates (e.g., larger than 25 μm), the risk of occlusions in the first and second fluid path components, the drive mechanism, and/or the cannula may be reduced. In some embodiments, a filter of a drug delivery device comprises an ion exchange filter.

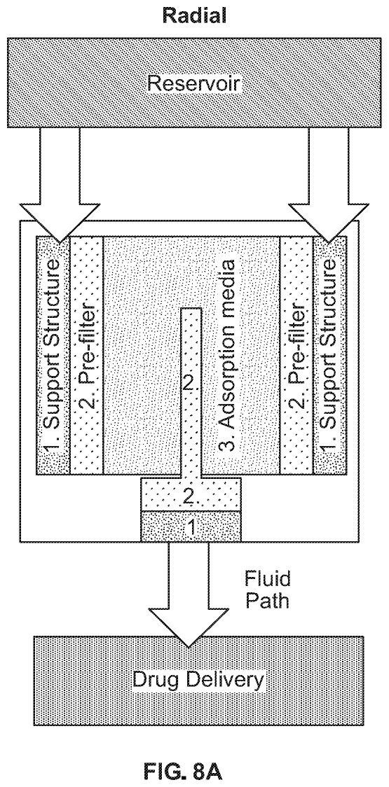

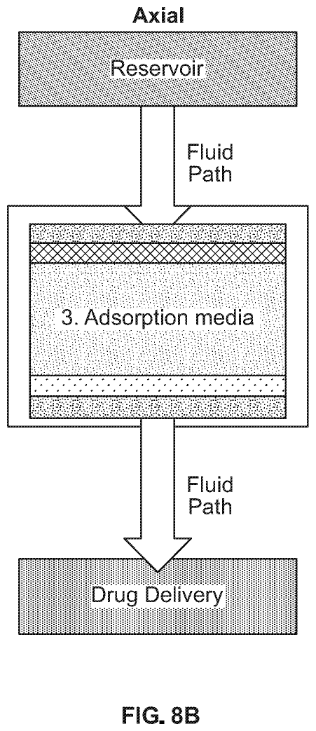

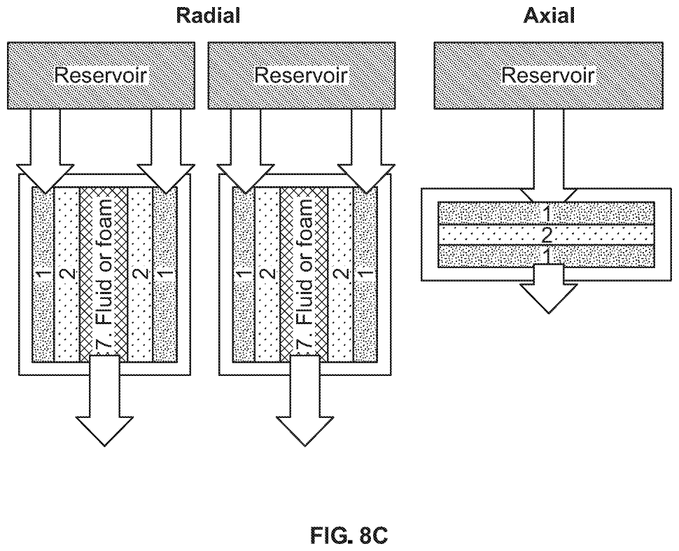

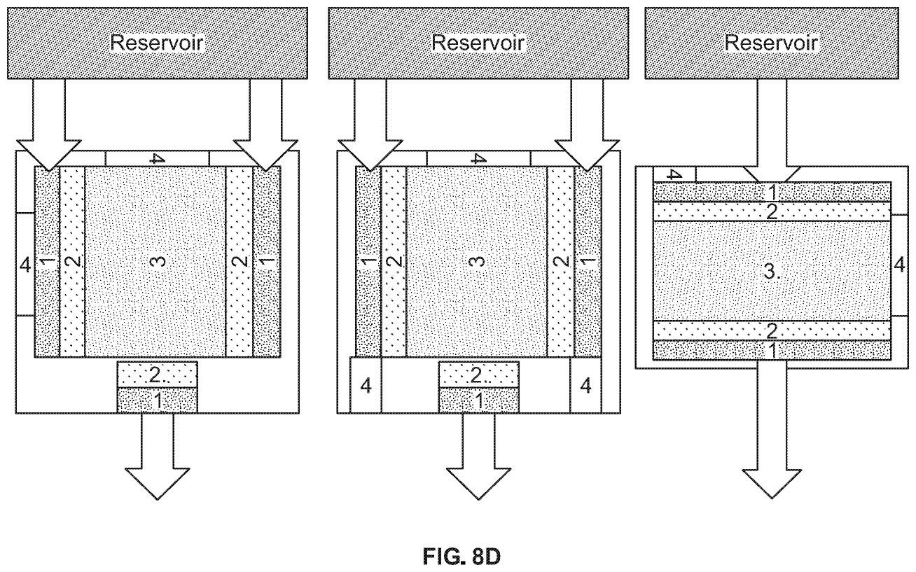

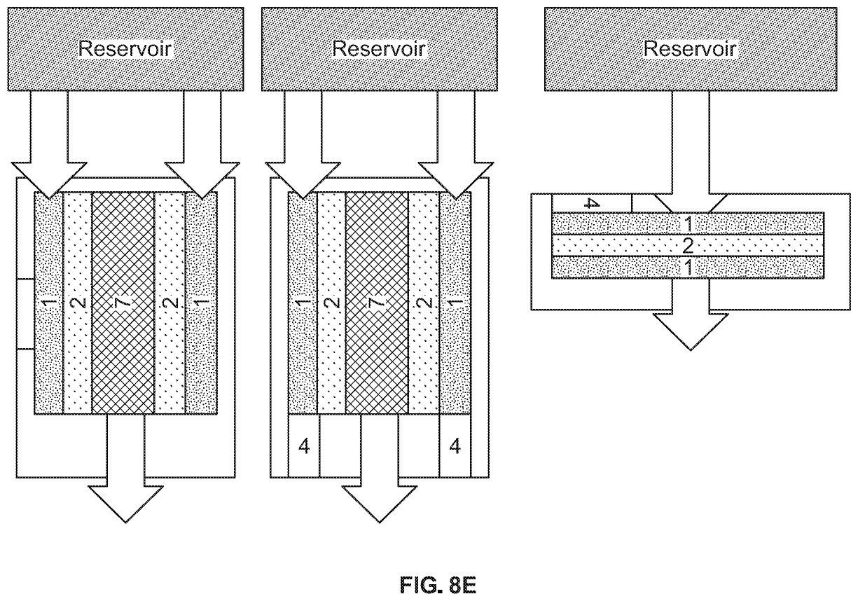

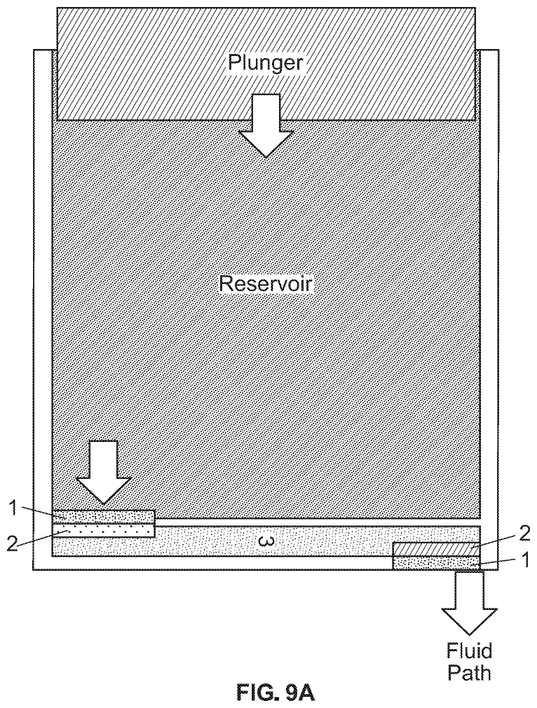

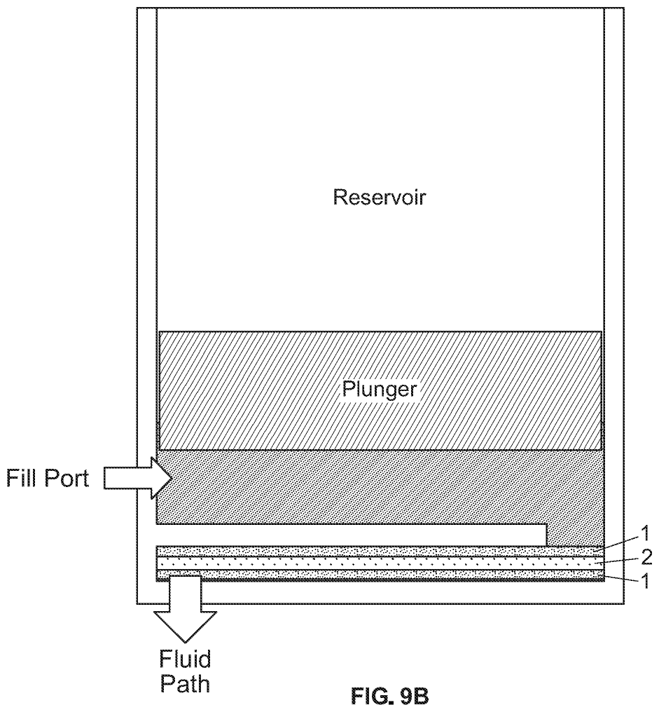

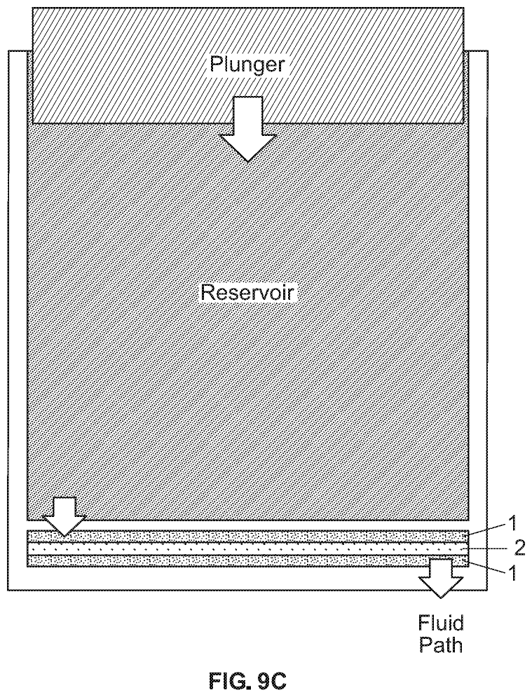

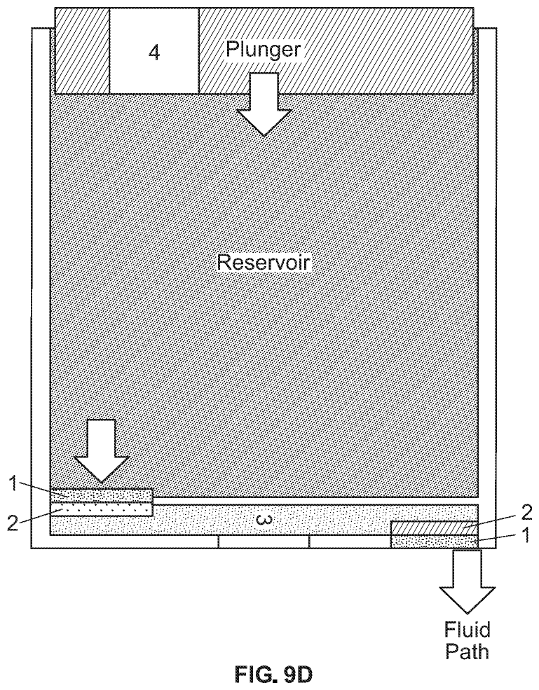

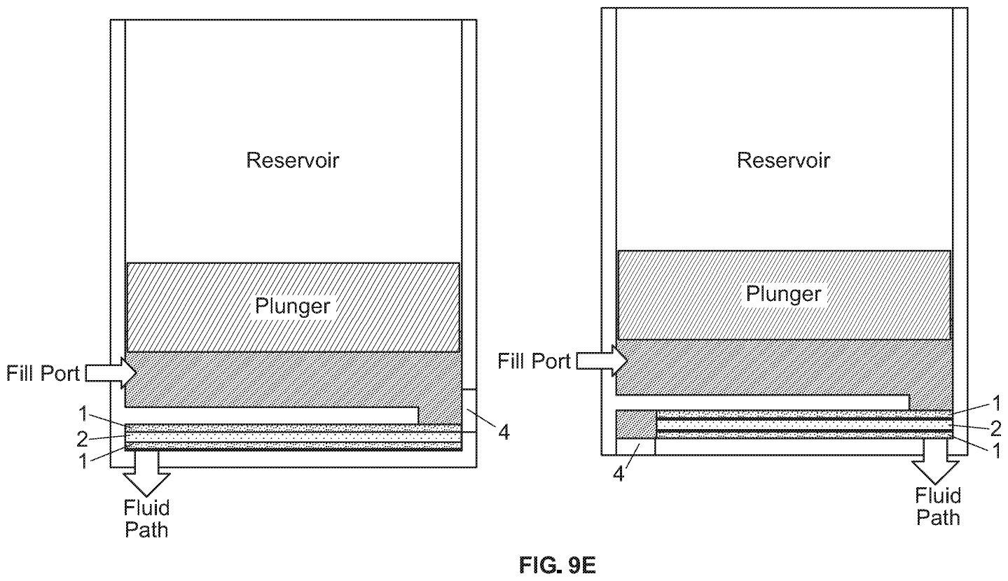

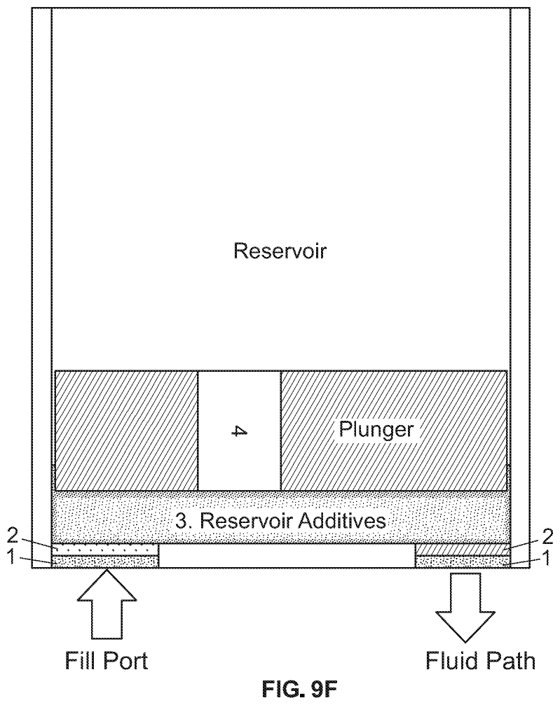

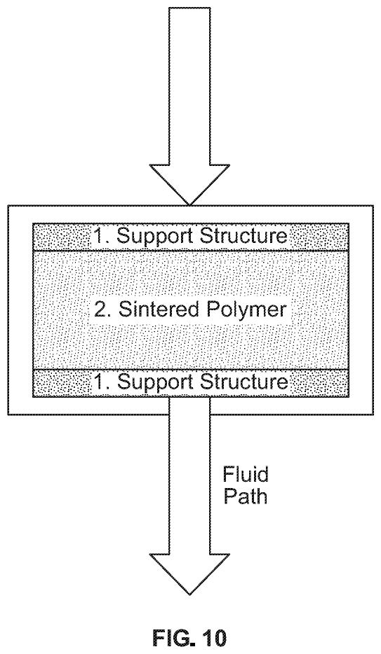

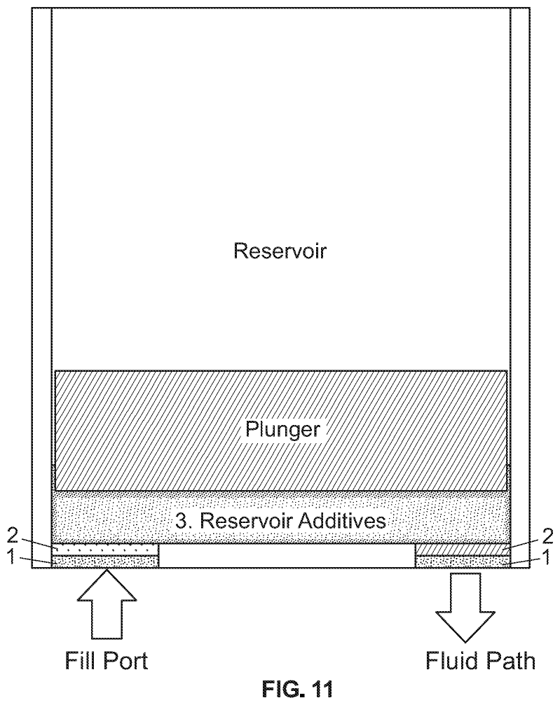

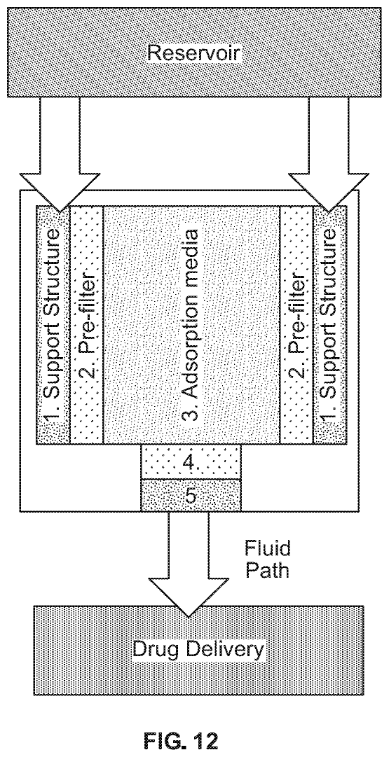

In some embodiments, a filter of a drug delivery device is arranged in a manner described in FIG. 8A-12.

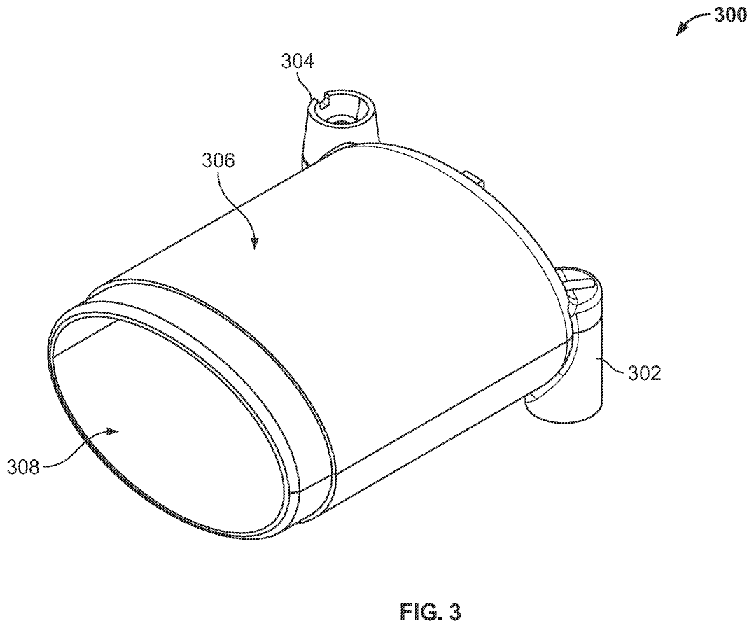

In one example, the drug delivery device includes a reservoir having a fixed volume that is operable to contain a liquid drug or liquid drug solution. FIG. 3 illustrates an example reservoir 300. In some examples, the reservoir 300 may correspond to the reservoir 126 of the system 100 of FIG. 1 or the reservoir 225 of the system 200 of FIGS. 2A, 2B. As shown in FIG. 3, the reservoir 300 may include a fluid inlet 302, a fluid outlet 304, and a fixed volume 306. In one example, the reservoir 300 may include an opening 308 allowing the reservoir 300 to be integrated with a plunger (e.g., plunger 202 as in system 200 of FIGS. 2A, 2B). In other examples, reservoir 300 may be separate from the drive mechanism used to expel the liquid drug from the drug delivery device (e.g., system 100 of FIG. 1). In such examples, the reservoir 300 may be fully enclosed with the exception of the inlet 302 and the outlet 304.

In one example, the fluid inlet 302 includes an inlet septum, an inlet fluid channel, and an inlet reservoir opening. In some examples, the inlet septum is operable to seal the inlet fluid channel from an exterior of the drug delivery device. The inlet reservoir opening may have an inlet volume positioned at an edge of the fixed volume 306 that is fluidly coupled to the inlet fluid channel. In some examples, the fluid inlet 302 is configured to be fluidly coupled to a drug injection port to receive the liquid drug to fill (or partially fill) the fixed volume 306. Likewise, the fluid outlet 304 includes an outlet reservoir opening and an outlet fluid channel. The outlet reservoir opening may have an outlet volume positioned at an edge of the fixed volume 306 that is fluidly coupled to the outlet fluid channel. In some examples, the fluid outlet 304 may be configured to be fluidly coupled to a cannula (e.g., cannulas 129, 233) to expel the liquid drug from the fixed volume 306. In other examples, the fluid inlet 302 and/or the fluid outlet 304 may be positioned differently. For example, the fluid outlet 304 may be positioned in the center of the fixed volume 306 or on a same side of the fixed volume 306 as the fluid inlet 302.

As described above, the liquid drug (or solution) may aggregate or precipitate over time in the reservoir 300, particularly as the liquid drug is being agitated with pump. In this context, “aggregation” may correspond to the unraveling and/or unfolding of liquid drug molecules and subsequent coiling into twisted fibrils or particles. In one example, the aggregation and/or precipitation of the liquid drug causes the generation of aggregates within the fixed volume 306. In some examples, these aggregates may be submicron particulates (e.g., fibrils). Pressure on the liquid drug may cause quicker aggregation, as it is forced through narrow fluid channels. Likewise, the liquid drug may gradually precipitate out of the liquid drug solution as the liquid drug ages. Preservatives (e.g., phenolic preservatives) may come out of the liquid drug solution over time. For example, over a 3-day period, the amount of preservatives in the liquid drug solution may decrease by about 30% (e.g., from about 3 mg to 2 mg). In some examples, these preservatives are submicron particulates. In some examples, polymers in the material of the reservoir 300 absorb a portion of these preservatives. However, these aggregates and preservatives (outside the liquid drug solution) may cause irritation and inflammation when delivered to the patient, may reduce the efficacy of the liquid drug, and/or may cause an occlusion within the fluid path of the pump. As such, it would be advantageous to utilize one or more filters configured to prevent liquid drug aggregates and preservatives from being delivered to the patient.

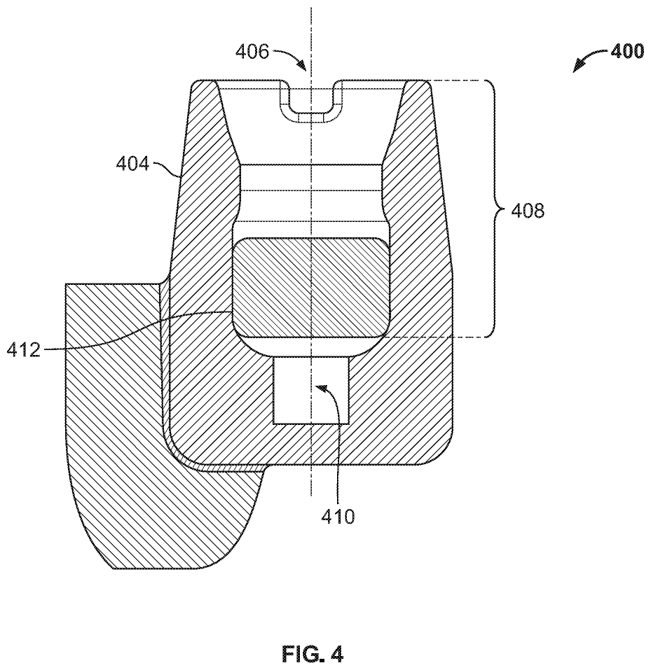

FIG. 4 illustrates a filter arrangement 400 of a liquid drug delivery device in accordance with aspects described herein. In one example, the filter arrangement 400 is operable for use with the systems 100, 200 of FIGS. 1 and 2A-2B.

As shown, the filter arrangement 400 includes a fluid outlet 404. In some examples, the fluid outlet 404 corresponds to the fluid outlet 304 of the reservoir 300 of FIG. 3. As shown, the fluid outlet 404 includes an outlet reservoir opening 406, an outlet volume 408, and an outlet fluid channel 410. In one example, the liquid drug stored in the reservoir enters the outlet fluid channel 410. The liquid drug is provided from the outlet fluid channel 410 to the outlet reservoir opening 406 via the outlet volume 408.

In one example, a filter 412 is positioned within the outlet volume 408. In some examples, the filter 412 corresponds to a filtration disk. The filter 412 may be operable to filter aggregates and absorb preservatives from the liquid drug as it passes through the outlet volume 408. In one example, a hard cannula (e.g., cannulas 129, 233 of FIGS. 1 and 2A, respectively) may be coupled to the outlet reservoir opening 406 after the filter 412. The hard cannula may provide a fluid pathway for the liquid drug after the liquid drug has passed through the filter 412.

In one example, the filter 412 has a diameter (or width) that is substantially the same as the diameter (or width) of the outlet volume 408. In some examples, the filter 412 is operable to fill at least a portion of the outlet volume 408 and collect particulate material as the liquid drug is expelled from the reservoir. In one example, the filter 412 may create a physical seal against the walls of the outlet volume 408 such that the liquid drug being expelled from the reservoir passes through the filter 412. In other examples, a sealant or other material (e.g., septum) may be used to create a seal between the filter 412 and the walls of the outlet volume 408.

In some embodiments, filter 412 comprises an absorption media, and optionally a pre-filter and a post filter. In some embodiments, filter 412 may further comprise a support structure. In some embodiments, filter 412 comprises an adsorption media positioned between a pre-filter and a post-filter. In some embodiments, filter 412 comprises a support structure, an adsorption media, a pre-filter, and a post-filter, wherein the adsorption media is positioned between the pre-filter and the post-filter. In some embodiments, a pre-filter or a post filter of filter 412 may have a maximum pore size that is smaller than the minimum particle size of an adsorption media. In some embodiments, a pre-filter or a post filter of filter 412 independently comprises cellulose acetate, PVDF, PES.

In one example, a filter of a drug delivery device is made from one or more porous filtration materials. In some examples, the porous filtration material may be a silica material, an alumina material, a polymeric material, or any combination thereof. For example, the porous filtration material of a filter of a drug delivery device may be a modified silica/alumina material. In some embodiments, an adsorption media comprises zeolite. In some embodiments, zeolite may have a controlled minimum particle size to prevent migration. In some embodiments, zeolite may be a zeolite membrane, a zeolite pellet, or a porous structure containing zeolite. In some embodiments, zeolite is ultra-stable zeolite Y.

In some embodiments, a filter of a drug delivery comprises one or more porous sintered polymers or a mixed media membranes. In some embodiments, a filter 412 comprises a one or more porous sintered polymers or a mixed media membranes and further comprises at least one support structure. In some embodiments, a filter 412 comprises a one or more porous sintered polymers or a mixed media membranes and further comprises at least two support structures. In some embodiments, zeolite may be integrated into one or more porous sintered polymers or mixed matrix membranes. In some embodiments, a porous sintered polymers comprises sintered polymer resin. In some embodiments, a sintered polymer resin is sintered polyvinylidene difluoride (PVDF), sintered polyether sulfone (PES), or sintered polyether ether ketone (PEEK). In some embodiments, sintered polymer resin is sintered polyvinylidene difluoride (PVDF).

In some examples, the porous filtration material of filter 412 may be rigid bare inorganic material In some examples, the porous filtration material of filter 412 may be a sponge-like polymeric material with a pore size of about 1 to 10 μm. In some embodiments, a filtration material of filter 412 may have a pore size of about 0.2 μm to 10.0 μm. In some embodiments, a filtration material of filter 412 may have a pore size of about 0.2 μm to 5.0 μm. In some embodiments, a filtration material of filter 412 may have a pore size of about 5.0 μm to 10.0 μm. In some embodiments, a filtration material of filter 412 may have a pore size of about 2.5 μm to 7.5 μm. In some embodiments, a filtration material of filter 412 may have a pore size of about 0.2 μm. In some embodiments, a filtration material of filter 412 may have a pore size of about 0.5 μm. In some embodiments, a filtration material of filter 412 may have a pore size of about 1.0 μm. In some embodiments, a filtration material of filter 412 may have a pore size of about 2.5 μm. In some embodiments, a filtration material of filter 412 may have a pore size of about 5.0 μm. In some embodiments, a filtration material of filter 412 may have a pore size of about 7.5 μm. In some embodiments, a filtration material of filter 412 may have a pore size of about 10.0 μm.

In one example, the porous filtration material may collect liquid drug aggregates or precipitants (e.g., insulin fibrils) that form over time in the liquid drug solution. In some examples, the porous filtration material may act to absorb preservatives in the liquid drug (or solution). The filter 412 may prevent undesired particulates from being delivered to the patient. In some embodiments, undesired particulates may comprise m-cresol, phenol, or methyl-hydroxy benzoate. In addition, by filtering out large particulates (e.g., larger than 25 μm), the risk of occlusions in the first and second fluid path components, the drive mechanism, and/or the cannula may be reduced.

While the example above describes a fluid outlet 404 having an integrated filter 412, it should be appreciated that in other examples the fluid inlet of the reservoir may be configured with a similar filter. For example, the reservoir of a liquid drug delivery device can be configured with an inlet filter and/or an outlet filter. In one example, the inlet filter is operable to fill the volume of the inlet reservoir opening and collect particulate material in the liquid drug entering via the inlet fluid channel prior to entering the reservoir.

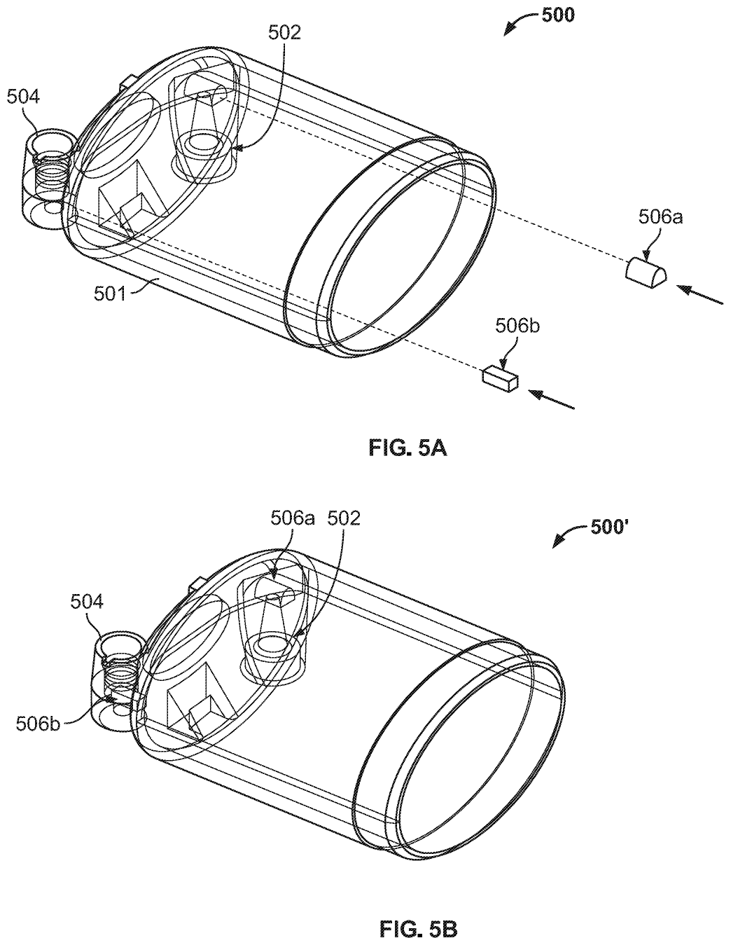

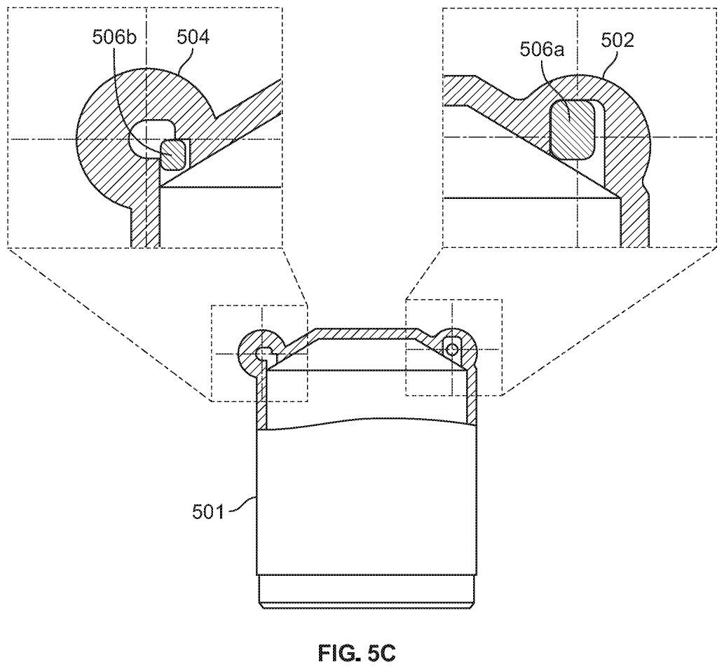

FIGS. 5A and 5B illustrate a liquid drug reservoir with a filter arrangement 500 of a liquid drug delivery device in accordance with aspects described herein. In one example, the filter arrangement 500 is operable for use with the systems 100, 200 of FIGS. 1 and 2A-2B.

As shown, the filter arrangement 500 includes a reservoir 501 having a fluid inlet 502 and a fluid outlet 504. In some examples, the reservoir 501 corresponds to the reservoir 300 of FIG. 3. FIG. 5A shows a first filter 506a and a second filter 506b prior to insertion into the reservoir 501. FIG. 5B shows the first filter 506a and the second filter 506b inserted into their respective positions with respect to the reservoir 501. In one example, a first filter 506a is inserted into the fluid inlet 502 and a second filter 506b is inserted into the fluid outlet 504. For example, as shown in FIG. 5B, the first filter 506a may be positioned within the inlet volume of the fluid inlet 502 and the second filter 506b may be positioned within the outlet volume of the fluid outlet 504. In another example, only filter 506b in fluid outlet 504 is used. In some examples, the filters 506a, 506b each correspond to a filtration plug (or filter plug). The first filter 506a may be operable to filter (or absorb) undesired particulates from the liquid drug as the liquid drug passes through the fluid inlet 502 into the reservoir 501. In some examples, the first filter 506a is further configured to prevent air from entering the reservoir 501. Likewise, the second filter 506b may be operable to filter (or absorb) aggregates and preservatives from the liquid drug as the liquid drug exits the reservoir 501 through the fluid outlet 504. In one example, a hard cannula (e.g., cannulas 129, 233 of FIGS. 1 and 2, respectively) is coupled to the outlet reservoir opening after the second filter 506b. The hard cannula may provide a fluid pathway for the liquid drug after the liquid drug has passed through the second filter 506b.

In one example, the first filter 506a has a diameter (or width) that is substantially the same as the diameter (or width) of the inlet volume of the fluid inlet 502. In some examples, the filter 506 is operable to fill at least a portion of the inlet volume and collect particulate material as the liquid drug enters the reservoir 501. In one example, the first filter 506a may create a physical seal against the walls of the inlet volume (or the fluid inlet 502) such that the liquid drug being provided to the reservoir 501 passes through the first filter 506a. In other examples, a sealant or other material (e.g., septum) may be used to create a seal between the first filter 506a and the walls of the inlet volume (or the fluid inlet 502).

The second filter 506b may, for example, have a diameter (or width) that is substantially the same as the diameter (or width) of the outlet volume of the fluid outlet 504. In some examples, the filter 506b is operable to fill at least a portion of the outlet volume and collect particulate material as the liquid drug is expelled from the reservoir 501. In an example, the second filter 506b may create a physical seal against the walls of the outlet volume (or the fluid outlet 504) such that the liquid drug being provided to the reservoir 501 passes through the second filter 506b. In other examples, a sealant or other material (e.g., septum) may be used to create a seal between the second filter 506b and the walls of the outlet volume (or the fluid outlet 504).

The first filter 506a and the second filter 506b may, for example, have substantially the same size (or volume). In some examples, the first filter 506a and the second filter 506b may be different sizes (or volumes). In an example, as shown in FIG. 5C, the first filter 506a of the fluid inlet 502 may be larger than the second filter 506b of the fluid outlet 504. However, in other examples (not shown), the second filter 506b of the fluid outlet 504 may be larger than the first filter 506a of the fluid inlet 502. In one example, the size (or volume) of each filter corresponds to the inlet/outlet volume. For example, the first filter 506a may have a volume that is substantially the same as the inlet volume of the fluid inlet 502. Likewise, second filter 506b may have a volume that is substantially the same as the outlet volume of the fluid outlet 504. In this example, the first filter 506a may substantially fill the inlet volume of the fluid inlet 502 and the second filter 506b may substantially fill the outlet volume of the fluid outlet 504. As such, the filters 506a, 506b can provide integrated filtration without reducing the fixed volume of the reservoir 501.

In some embodiments, filters 506a, 506b comprise an absorption media, and optionally a pre-filter and a post filter. In some embodiments, filters 506a, 506b may further comprise a support structure. In some embodiments, filters 506a, 506b comprise an adsorption media positioned between a pre-filter and a post-filter. In some embodiments, filters 506a, 506b comprise a support structure, an adsorption media, a pre-filter, and a post-filter, wherein the adsorption media is positioned between the pre-filter and the post-filter. In some embodiments, a pre-filter or a post filter of filters 506a, 506b may have a maximum pore size that is smaller than the minimum particle size of an adsorption media. In some embodiments, a pre-filter or a post filter of filters 506a, 506b independently comprise cellulose acetate, PVDF, PES.

In one example, each of the filters 506a, 506b is made from one or more porous filtration materials. In some examples, the porous filtration material may be a silica material, an alumina material, a polymeric material, or any combination thereof. For example, the porous filtration material of the filters 506a, 506b may be a modified silica/alumina material. In some embodiments, an adsorption media comprises zeolite. In some embodiments, zeolite may have a controlled minimum particle size to prevent migration. In some embodiments, zeolite may be a zeolite membrane, a zeolite pellet, or a porous structure containing zeolite. In some embodiments, zeolite is ultra-stable zeolite Y.

In some examples, the porous filtration material of the filters 506a, 506b may be rigid bare inorganic material. In some examples, the porous filtration material of the filters 506a, 506b may be a sponge-like polymeric material with a pore size of about 1 micrometer (μm) to about 10 μm. In one example, the porous filtration material may filter or collect the liquid drug aggregates or precipitants (e.g., insulin fibrils) that form over time in the liquid drug solution. In some examples, the porous filtration material may act to absorb preservatives in the liquid drug (or solution). For example, the preservatives in the liquid drug may have an affinity to the porous filtration material of the filters 506a, 506b. Given the molecular size of the preservatives, the preservatives can be absorbed by the filters 506a, 506b without clogging or blocking the pores of the filters 506a, 506b. In one example, filters 506a, 506b may use the same filtration material configuration; however, in other examples, the filters 506a, 506b may have different material configurations. The filters 506a, 506b may prevent undesired particulates from being delivered to the patient. In addition, by filtering out large particulates (e.g., larger than 25 μm), the risk of occlusions in the cannula fluidly coupled to the reservoir 501 may be reduced.

In some embodiments, a filter of filters 506a, 506b comprises one or more porous sintered polymers or a mixed media membranes. In some embodiments, a filter of filters 506a, 506b comprises a one or more porous sintered polymers or a mixed media membranes and further comprises at least one support structure. In some embodiments, filters 506a, 506b comprise a one or more porous sintered polymers or a mixed media membranes and further comprises at least two support structures. In some embodiments, zeolite may be integrated into one or more porous sintered polymers or mixed matrix membranes. In some embodiments, a porous sintered polymers comprises sintered polymer resin. In some embodiments, a sintered polymer resin is sintered polyvinylidene difluoride (PVDF), sintered polyether sulfone (PES), or sintered polyether ether ketone (PEEK). In some embodiments, sintered polymer resin is sintered polyvinylidene difluoride (PVDF).

In some examples, the porous filtration material of filters 506a, 506b may be rigid bare inorganic material In some examples, the porous filtration material of filters 506a, 506b may be a sponge-like polymeric material with a pore size of about 1 to 10 μm. In some embodiments, a filtration material of filters 506a, 506b may have a pore size of about 0.2 μm to 10 μm. In some embodiments, a filtration material of filters 506a, 506b may have a pore size of about 0.2 μm to 5.0 μm. In some embodiments, a filtration material of filters 506a, 506b may have a pore size of about 5.0 μm to 10.0 μm. In some embodiments, a filtration material of filters 506a, 506b may have a pore size of about 2.5 μm to 7.5 μm. In some embodiments, a filtration material of filters 506a, 506b may have a pore size of about 0.2 μm. In some embodiments, a filtration material of filters 506a, 506b may have a pore size of about 0.5 μm. In some embodiments, a filtration material of filters 506a, 506b may have a pore size of about 1.0 μm. In some embodiments, a filtration material of filters 506a, 506b may have a pore size of about 2.5 μm. In some embodiments, a filtration material of filters 506a, 506b may have a pore size of about 5.0 μm. In some embodiments, a filtration material of filters 506a, 506b may have a pore size of about 7.5 μm. In some embodiments, a filtration material of filters 506a, 506b may have a pore size of about 10.0 μm.

While the example above describes a fluid inlet 502 having an integrated filter 506a and a fluid outlet 504 having an integrated filter 506b, it should be appreciated that in other examples the filter 506a and/or the filter 506b may be optional. For example, the reservoir 501 may be configured with just the second filter 506b positioned within the fluid outlet 504.

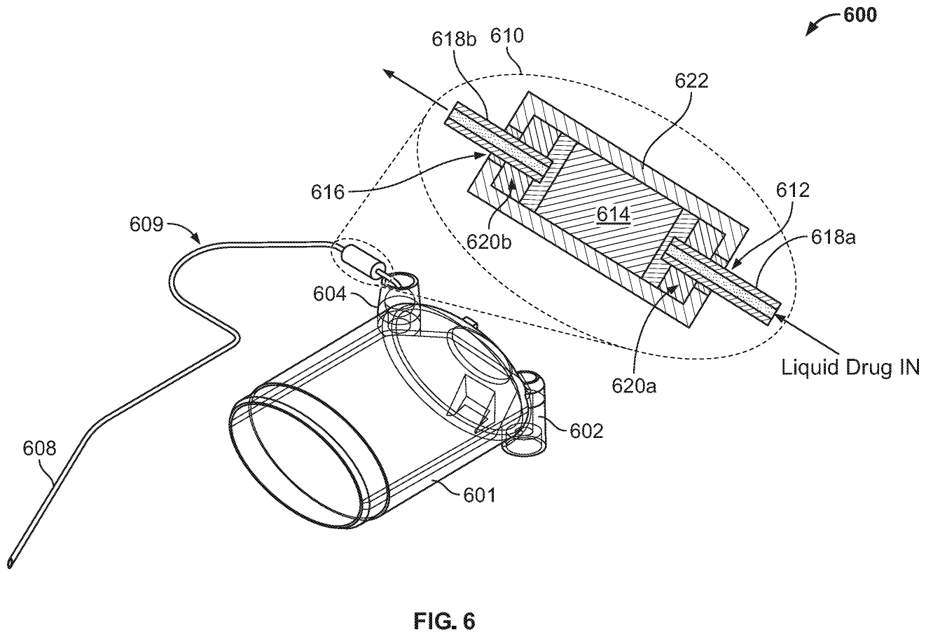

FIG. 6 illustrates a filter arrangement 600 of a liquid drug delivery device in accordance with aspects described herein. In one example, the filter arrangement 600 is operable for use with the systems 100, 200 of FIGS. 1 and 2A-2B.

As shown, the filter arrangement 600 includes a reservoir 601 having a fluid inlet 602 and a fluid outlet 604. In one example, the reservoir 601 corresponds to the reservoir 300 of FIG. 3. The reservoir 601 may, for example, have a fixed volume that is operable to contain a liquid drug or liquid drug solution. The fluid outlet 604 may include an outlet reservoir opening and an outlet fluid channel (not shown in this example). In some examples, a hard cannula or needle 608 is coupled to the outlet reservoir opening of the fluid outlet 604 via a fluid pathway 609. The needle 608 may be operable to receive the liquid drug when the liquid drug is expelled from the reservoir 601.

The filter arrangement 600 may, for example, include a filtration module 610 having an inlet 612, a filter 614, and an outlet 616. The filtration module 610 may be placed anywhere along the fluid path 609 within a housing of the system 100, 200. In some examples, a first segment 618a of the fluid pathway 609 may be operable to fluidly couple to the inlet 612 of the filtration module 610 to the fluid outlet 604 of the reservoir 601. The first segment 618a may provide the liquid drug to the filtration module 610 after the liquid drug has been expelled from the reservoir 601. In one example, the inlet 612 includes a pierceable inlet septum 620a, and the first segment 618a may be configured to pierce the inlet septum 620a. The pierceable inlet septum 620a may, for example, be a silicon septum, a septum formed from one or more other materials, or the like. Likewise, the fluid pathway 609 may have a second segment 618b operable to fluidly couple the outlet 616 of the filtration module 610 to fluid path 609. In one example, the outlet 616 includes a pierceable outlet septum 620b and the second segment 618b of the cannula 608 is configured to pierce the outlet septum 620b. The pierceable outlet septum 620b may be a silicon septum, a septum formed from one or more other materials, or the like. In some examples, the filter 614 and the pierceable septums 620a, 620b are included in a filter enclosure 622. The second segment 618b of the fluid pathway 609 may provide the liquid drug to the hard cannula or needle 608 after the liquid drug has passed through the filter 614.

The filter 614 is formed from a porous filtration material. The porous filtration material may be a porous silica material, a porous alumina material, a porous polymeric material, or a combination thereof. For example, the porous filtration material of the filter 614 may be a modified silica/alumina material. In some examples, the porous filtration material of the filter 614 may be rigid bare inorganic material. In some examples, the porous filtration material of the filter 614 may be a sponge-like polymeric material with a pore size of approximately 1 μm to approximately 10 μm. In addition, the size of all of the pores may not all be uniform. The porous filtration material may, for example, collect the liquid drug aggregates or precipitants (e.g., insulin fibrils) that form over time in the liquid drug solution. In some examples, the porous filtration material may act to absorb preservatives in the liquid drug (or solution). The filter 614 may prevent undesired particulates from being delivered to the patient. In addition, by filtering out large particulates (e.g., larger than 25 μm), the risk of occlusions in the cannula 608 may be reduced. The filter 614 may also be formed to filter out small and medium particulates, such as 5 μm, 10 μm, 15 μm or the like.

In some embodiments, filter 614 comprises an absorption media, and optionally a pre-filter and a post filter. In some embodiments, filter 614 may further comprise a support structure. In some embodiments, filter 614 comprises an adsorption media positioned between a pre-filter and a post-filter. In some embodiments, filter 614 comprises a support structure, an adsorption media, a pre-filter, and a post-filter, wherein the adsorption media is positioned between the pre-filter and the post-filter. In some embodiments, a pre-filter or a post filter of filter 614 may have a maximum pore size that is smaller than the minimum particle size of an adsorption media. In some embodiments, a pre-filter or a post filter of filter 614 independently comprises cellulose acetate, PVDF, PES.

In one example, a filter of a drug delivery device is made from one or more porous filtration materials. In some examples, the porous filtration material may be a silica material, an alumina material, a polymeric material, or any combination thereof. For example, the porous filtration material of a filter of a drug delivery device may be a modified silica/alumina material. In some embodiments, an adsorption media comprises zeolite. In some embodiments, zeolite may have a controlled minimum particle size to prevent migration. In some embodiments, zeolite may be a zeolite membrane, a zeolite pellet, or a porous structure containing zeolite. In some embodiments, zeolite is ultra-stable zeolite Y.

In some embodiments, a filter of a drug delivery comprises one or more porous sintered polymers or a mixed media membranes. In some embodiments, a filter 614 comprises a one or more porous sintered polymers or a mixed media membranes and further comprises at least one support structure. In some embodiments, a filter 614 comprises a one or more porous sintered polymers or a mixed media membranes and further comprises at least two support structures. In some embodiments, zeolite may be integrated into one or more porous sintered polymers or mixed matrix membranes. In some embodiments, a porous sintered polymers comprises sintered polymer resin. In some embodiments, a sintered polymer resin is sintered polyvinylidene difluoride (PVDF), sintered polyether sulfone (PES), or sintered polyether ether ketone (PEEK). In some embodiments, sintered polymer resin is sintered polyvinylidene difluoride (PVDF).

In some examples, the porous filtration material of filter 614 may be rigid bare inorganic material In some examples, the porous filtration material of filter 614 may be a sponge-like polymeric material with a pore size of about 1 to 10 μm. In some embodiments, a filtration material of filter 614 may have a pore size of about 0.2 μm to 10.0 μm. In some embodiments, a filtration material of filter 614 may have a pore size of about 0.2 μm to 5.0 μm. In some embodiments, a filtration material of filter 614 may have a pore size of about 5.0 μm to 10.0 μm. In some embodiments, a filtration material of filter 614 may have a pore size of about 2.5 μm to 7.5 μm. In some embodiments, a filtration material of filter 614 may have a pore size of about 0.2 μm. In some embodiments, a filtration material of filter 614 may have a pore size of about 0.5 μm. In some embodiments, a filtration material of filter 614 may have a pore size of about 1.0 μm. In some embodiments, a filtration material of filter 614 may have a pore size of about 2.5 μm. In some embodiments, a filtration material of filter 614 may have a pore size of about 5.0 μm. In some embodiments, a filtration material of filter 614 may have a pore size of about 7.5 μm. In some embodiments, a filtration material of filter 614 may have a pore size of about 10.0 μm.

In one example, the porous filtration material may collect liquid drug aggregates or precipitants (e.g., insulin fibrils) that form over time in the liquid drug solution. In some examples, the porous filtration material may act to absorb preservatives in the liquid drug (or solution). The filter 614 may prevent undesired particulates from being delivered to the patient. In some embodiments, undesired particulates may comprise m-cresol, phenol, or methyl-hydroxy benzoate. In addition, by filtering out large particulates (e.g., larger than 25 μm), the risk of occlusions in the first and second fluid path components, the drive mechanism, and/or the cannula may be reduced.

While the example above describes a filtration module 610 fluidly coupled to the fluid outlet 604, it should be appreciated that in other examples the fluid inlet 602 of the reservoir 601 may be fluidly coupled to a filtration module similar to that of filtration module 610. In other words, the reservoir 601 can be fluidly coupled to an inlet filtration module and/or an outlet filtration module.

As described above, the liquid drug (or solution) may aggregate or precipitate over time in the reservoir. In some examples, the reservoir is made from one or more hydrophobic surfaces (e.g., polypropylene, Teflon, etc.). As such, when the user (e.g., patient) transfers the liquid drug into the reservoir, the liquid drug is exposed to hydrophobic surfaces which may initiate aggregation. In addition, the liquid drug may be exposed to hydrophobic surfaces when enroute from the reservoir to the distal tip of the cannula. For example, the liquid drug solution stored in the reservoir may contain protective diluent components (e.g., m-cresol or the like) that are removed from the liquid drug solution enroute to the distal tip of the cannula. In some examples, shortening the fluidic path between the reservoir and the distal tip of the cannula may reduce the effects of this exposure. However, shortening the fluidic path may increase the likelihood of wound immune response components (e.g., IgG and IgE) migrating from the distal tip of the cannula back into the reservoir. As such, it may be advantageous to utilize a flow director to prevent immunoglobulins, immune response components, and/or other undesired particulates from migrating back to the reservoir.

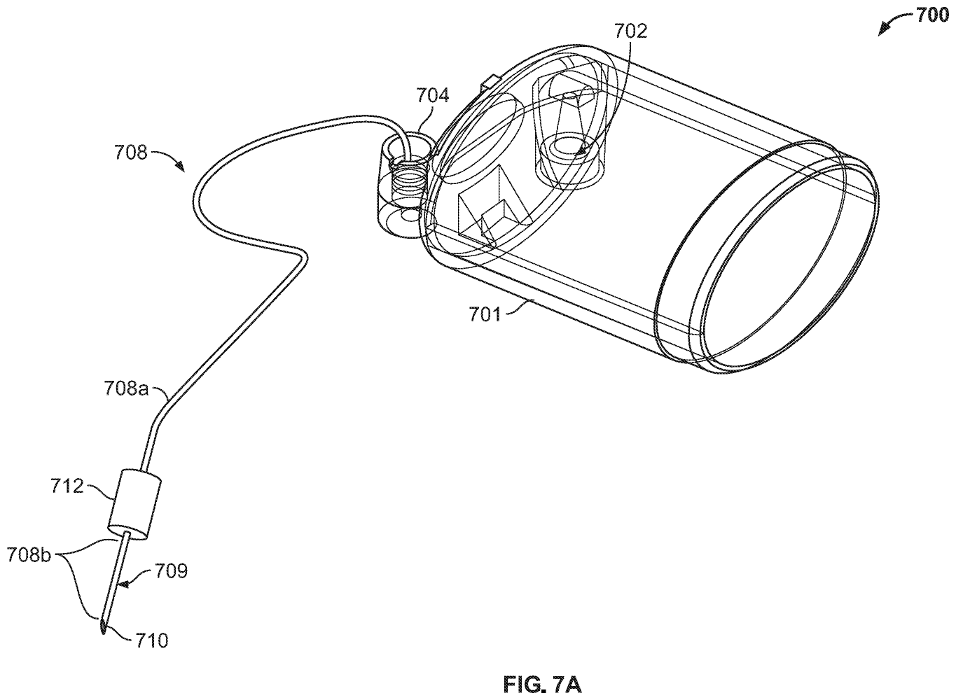

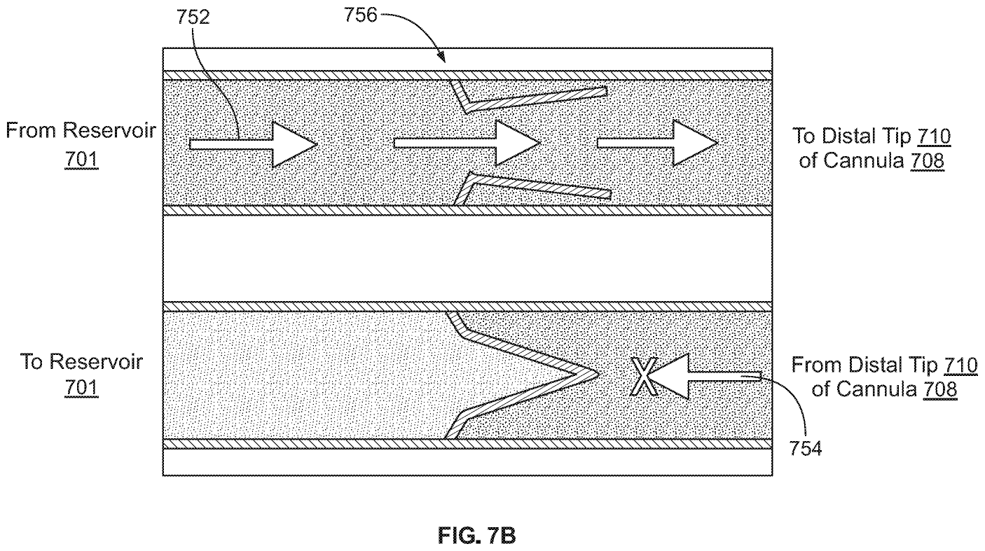

FIG. 7A illustrates a flow director arrangement 700 of a liquid drug delivery device in accordance with aspects described herein. In one example, the flow director arrangement 700 is operable for use with the systems 100, 200 of FIGS. 1 and 2A-2B. As shown, the flow director arrangement 700 may include a reservoir 701 having a fluid inlet 702 and a fluid outlet 704. In some examples, the reservoir 701 corresponds to the reservoir 300 of FIG. 3. In one example, a fluid pathway 708 is coupled to the fluid outlet 704. The fluid pathway 708 may be a tube or channel that provides the liquid drug to a flow director device 712 after the liquid drug has been expelled from the reservoir 701. In some examples, the fluid pathway 708 may have a first segment 708a and a second segment 708b. The second segment 708b may terminate with a cannula 709. The cannula 709 may be made from a hydrophobic material (e.g., Teflon, steel, fluorinated ethylene propylene (FEP), etc.). In one example, the cannula 709 includes a distal tip 710 that is deployable into the patient. As such, the cannula 709 may form a portion of the fluid path 708 and enable liquid drug from the reservoir 701 to be delivered to the patient.

The fluid flow director arrangement 700 may also include a flow director device 712, which is fluidly coupled between the reservoir 701 and the distal tip 710 of the cannula 709, and in some examples within a pump housing of system 100, 200. In some examples, the flow director device 712 is configured to regulate flow in a single direction (e.g., from the reservoir 701 to the distal tip 710) to encourage fluid isolation between the reservoir 701 and the distal tip 710 of the cannula 708. In one example, the flow director device 712 may be disposed within the cannula 709. For example, a first segment 708a of the fluid pathway 708 may couple the flow director device 712 to the fluid outlet 704 of the reservoir 701. Likewise, a second segment 708b of the fluid pathway 708 may couple the flow director device 712 to the cannula 709. In one example, the flow director device 712 may be positioned as close to the distal tip 710 as possible (e.g., within several millimeters) to minimize the length of the back flow path from the distal tip 710. In other words, the flow director device 712 may be positioned such that the first segment 708a is longer than the second segment 708b. For example, the flow director device 712 may be positioned within or as part of the cannula 709. The flow director device 712 may reduce liquid drug aggregation in the cannula 709 and/or at the interface of the distal tip 710 with the surrounding tissue of the patient. In addition, the flow director device 712 may reduce the occurrence of occlusions at the distal tip 710 of the cannula 709.

In one example, the flow director device 712 includes a duckbill value. The duckbill valve may be configured with a low cracking pressure that allows the liquid drug to pass through the duckbill valve such that the liquid drug can be expelled from the reservoir 701 and delivered to the patient at relatively low flow rates. For example, as illustrated in FIG. 7B, the liquid drug (or solution) may flow in a first direction 752 when expelled from the reservoir 701. The flow rate of the liquid drug in the first direction 752 may provide a cracking pressure that exceeds a threshold of the duckbill valve 756, enabling the liquid drug to flow from the reservoir 701 to the distal tip 710. Immunoglobulins, immune response components, and other undesired particulates may attempt to flow in a second direction 754 back through the duckbill valve 756. However, the duckbill valve 756 may be configured to block flow in the second direction 754, preventing the undesired components from flowing back toward the reservoir 701.

In one example, the cracking pressure of the duckbill valve 756 is selected such that a hexamer form of the liquid drug (e.g., insulin) can pass to the distal tip 710 of the hard cannula 708. In other examples, the low cracking pressure of the duckbill valve 756 can extend the longevity of the hexamer form of the liquid drug.

As described above, improved drug delivery devices with incorporated filtration are provided herein. In at least one example, the drug delivery device includes at least one filter configured to collect particulate material as the liquid drug (or solution) is expelled from the reservoir. In one example, the at least one filter is a filter plug operable to fill the volume of an outlet opening and/or an inlet opening of the reservoir. In some examples, the at least one filter is included in a filtration module that is fluidly coupled to the reservoir.

In some examples, the filter arrangements 400, 500, 600 of FIGS. 4-6 or the flow director arrangement 700 of FIG. 7 can include a flow indicator that provides a flow status to the user (e.g., patient). For example, a flexible strip included in the liquid drug delivery device may indicate to the user that the device is operating with a normal flow, the device is clogged, an occlusion has occurred, etc. The flow status may be provided to the user via a user interface of the controller 102 of FIG. 1 or the controller 221 of FIG. 2A. In other examples, the flow status is provided to the user via another user device, such as a smartphone or a personal diabetes manager (PDM).

As used herein, the algorithms or computer applications that manage blood glucose levels and insulin therapy may be referred to as an “artificial pancreas” algorithm-based system, or more generally, an artificial pancreas (AP) application. An AP application may be programming code stored in a memory device and that is executable by a processor, controller or computer device.

The techniques described herein for a drug delivery system (e.g., the system 100, the system 200, or any components thereof) may be implemented in hardware, software, or any combination thereof. Any component as described herein may be implemented in hardware, software, or any combination thereof. For example, the systems 100, 200 or any components thereof may be implemented in hardware, software, or any combination thereof. Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

Some examples of the disclosed devices may be implemented, for example, using a storage medium, a computer-readable medium, or an article of manufacture which may store an instruction or a set of instructions that, if executed by a machine (i.e., processor or controller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosed subject matter were described above. It is, however, expressly noted that the present disclosed subject matter is not limited to those examples, but rather the intention is that additions and modifications to what was expressly described herein are also included within the scope of the disclosed subject matter. Moreover, it is to be understood that the features of the various examples described herein were not mutually exclusive and may exist in various combinations and permutations, even if such combinations or permutations were not made express herein, without departing from the spirit and scope of the disclosed subject matter. In fact, variations, modifications, and other implementations of what was described herein will occur to those of ordinary skill in the art without departing from the spirit and the scope of the disclosed subject matter. As such, the disclosed subject matter is not to be defined only by the preceding illustrative description.

Program aspects of the technology may be thought of as “products” or “articles of manufacture” typically in the form of executable code and/or associated data that is carried on or embodied in a type of machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of example examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A drug delivery device, comprising:
a reservoir operable to contain a liquid drug;
a fluid outlet including an outlet reservoir opening and an outlet fluid channel, wherein the outlet reservoir opening has a volume and is fluidly coupled to the outlet fluid channel; and
an outlet filter plug operable to fill the volume of the outlet reservoir opening and collect particulate material as the liquid drug is expelled from the reservoir.

2. The drug delivery device of claim 1, wherein the outlet filter plug comprises: a porous material having a pore size of about 1 micrometer to about 10 micrometers.

3. The drug delivery device of claim 1, wherein the outlet filter plug comprises: a porous material having a pore size of about 1 micrometer to about 10 micrometers.

4. The drug delivery device of claim 1, wherein the outlet filter plug is operable to absorb phenolic preservatives in the liquid drug.

5. The drug delivery device of claim 1, wherein the outlet filter plug is operable to collect precipitant in the liquid drug.

6. The drug delivery device of claim 1, wherein the outlet filter plug comprises: a porous silica material, a porous alumina material, a porous polymeric material, or a combination thereof.

8. The drug delivery device of claim 1, wherein the outlet filter plug comprises an adsorption layer.

9. The drug delivery device of claim 8, wherein the outlet filter plug comprises an adsorption layer.

10. The drug delivery device of claim 9, wherein the adsorption layer comprises zeolite.

11. The drug delivery device of claim 10, wherein the adsorption layer comprises a zeolite membrane, a zeolite pellet, or a porous structure containing zeolite.

12. The drug delivery device of claim 10, wherein the zeolite is ultra-stable zeolite Y.

13. The drug delivery device of any of claim 9, wherein the adsorption layer further comprises a chelator.

14. The drug delivery device of claim 13, where the chelator is EDTA.

15. The drug delivery device of claim 1, further comprising:
a hard cannula or needle coupled to the outlet fluid channel after the outlet filter plug, wherein the hard cannula or needle is a fluid pathway for the liquid drug after the liquid drug has passed through the outlet filter plug.

16. The drug delivery device of claim 15, further comprising:
a duckbill valve disposed within the hard cannula or needle, wherein the duckbill valve has a cracking pressure operable to allow a hexamer form of the liquid drug to pass to the distal tip of the hard cannula or needle.

17. The drug delivery device of claim 16, wherein the duckbill valve is positioned proximate a distal end of the hard cannula.

18. The drug delivery device of claim 1, further comprising:
a fluid inlet including an inlet septum, an inlet fluid channel, and an inlet reservoir opening, wherein the inlet reservoir opening has an inlet volume and is fluidly coupled to the reservoir; and
an inlet filter plug operable to fill the volume of the inlet reservoir opening and collect particulate material in the liquid drug entering via the inlet fluid channel prior to entering the reservoir.

19. The drug delivery device of claim 18, the fluid inlet further comprising:
an inlet septum operable to seal the inlet fluid channel from an exterior of the drug delivery device.

20. The drug delivery device of claim 18, wherein the inlet filter plug is operable to absorb phenolic preservatives in the liquid drug.

21. The drug delivery device of claim 18, wherein the inlet filter plug is operable to collect precipitant in the liquid drug.

22. The drug delivery device of claim 18, further comprising:
a hard cannula coupled to the outlet fluid channel after the outlet filter plug, wherein the hard cannula or needle is operable to receive the liquid drug after the liquid drug has passed through the outlet filter plug.

23. The drug delivery device of claim 18, further comprising:
a duckbill valve disposed within the hard cannula or needle, wherein the duckbill valve has a cracking pressure operable to allow a hexamer form of the liquid drug to pass to the distal tip of the hard cannula.

24. A drug delivery device, comprising:
a reservoir operable to contain a liquid drug;
a fluid outlet including an outlet reservoir opening and an outlet fluid channel;
a filtration module having an inlet, a filter and an outlet; and
a needle coupled to the outlet fluid channel and operable to receive the liquid drug when the liquid drug is expelled from the reservoir, wherein:
the needle has a first segment operable to fluidly couple to the inlet and a second segment operable to couple the outlet of the filtration module, and
the filter is comprised of a porous material, wherein the porous material is a porous silica material, a porous alumina material, a porous polymeric material, or a combination thereof.

25. The drug delivery device of claim 24, wherein the porous material has a pore size of about 1 micrometer to about 10 micrometers.

26. The drug delivery device of claim 24, wherein the inlet of the filtration module comprises a pierceable inlet septum.

27. The drug delivery device of claim 24, wherein the outlet of the filtration module comprises a pierceable outlet septum.

28. The drug delivery device of claim 27, wherein the needle has a first segment operable to pierce the inlet septum and a second segment operable to pierce the outlet septum.
